(19) United States
(12) Patent Application Publication
(10) Pub. No.: US 2008/0101998 A1
(43) Pub. Date: May 1, 2008

(54) Air purification system and apparatus

(76) Inventor: Clayton Armstrong, Paris, MO (US)

Correspondence Address: Paul M. Denk;Suite 170, 763 S. New Ballas Rd., St. Louis, MO, 63141, US

(21) Appl. No.: 11/975,248

(22) Filed: Oct. 18, 2007

Related U.S. Application Data
(60) Provisional application No. 60/854,288, filed on Oct. 25, 2006.

(51) Int. Cl. A61L 2/10

(52) U.S. Cl. 422/121

(57) ABSTRACT

An air purification system and apparatus including a housing, the housing incorporating various deflectors therein, and which support one or more blowers, for drawing contaminated air in through and intake grill and filter, and passing it through the housing, and its deflectors, into a confined space in proximity with one or more ultraviolet lamp, to attain air purification, before the air is forced by the deflectors out of an exhaust outlet for the housing and back into the room as purified air.

### CROSS REFERENCE TO RELATED APPLICATION

This non-provisional application claims priority to provisional application Ser. No. 60/854,288 filed on Oct. 25, 2006.

### FIELD OF INVENTION

This invention relates to means for purification of air, and more specifically relates to an air filtering and purification system wherein air is drawn into an apparatus and condensed to an atmosphere of ambient air subjected to ultraviolet light wherein the ozone has a tendency to substantially reduce any bacteria or biological agents, or airborne pollutants, from the air before it is discharged back into a room or other living quarters.

### BACKGROUND OF THE INVENTION

As is well known, air pollutants within a living environment can be deleterious to the occupants, and whether such environment may include a residence, home, business quarters, a hospital room, or even other confined spaces, such as on an airline, such pollutants can cause health problems to any occupants when exposed to such over a period of time. Thus, if a means could be provided to eliminate or substantially reduce any such airborne infectious matter or pollutants, so that the breathable air is more purified, this can provide a substantially enhanced living environment, regardless whether it is in a home, commercial, or other setting.

Others have given consideration to the treatment of ambient air, through air purification systems, in order to attempt to solve the problems relating to such exposure. For example, in the United States patent to Jones, U.S. Pat. No. 5,925,320, an Air Purification System is disclosed. This provides for a generalized type of purification system, that incorporates a housing, a motor for drawing air into the housing, a filter through which the incoming air flows, and then the blower forces the air into a circuitous path, where it is subjected to UVC purification process, which apparently has the benefit of removing some of the contaminants from the circulating air. As can be noted, the Jones system includes a curved type of passageway, which is relatively narrow at its intake end, but widens at its outlet end, and where the purified air is discharged into the room. At its narrowest point, of the curved passageway, there is located a UVC source, to which the circulating air is exposed, and which attempts to create an ozone, or photozone molecules, that have the effect of purifying the air, circulating therethrough.

The current invention, though, is designed to enhance the efficiency of reducing airborne pollutants, by drawing air directly into a housing, forcing it upwardly into an area where substantial ozone having high energy level is generated, and therein destroys any pathogens, while having the capacity of immediately forcing the purified air directly out of the housing and into the living environment.

### SUMMARY OF THE INVENTION

This invention contemplates the fabrication of an air purification system, all embodied within a single housing, having a filtered intake at its lower end, and a discharge of the purified air at its upper or top segment. Within the housing, there is provided means for generating an ozone region, when the drawn air is passed by one or a series of ultraviolet bulbs, illuminated, so that the passing air will be subjected directly to ultraviolet illumination. The filter can filter out and eliminate up to seventy-five to eighty percent of the airborne pollutants, whether it be bacterial, or other airborne pollution. To achieve this, a series of dividers and deflectors are located within the housing, and direct the drawn in air in a direction upwardly towards a confined and constricted space, perhaps not more than fourteen inches by two to three inches in width, all within the region of the located bulbs, so that the air is directly exposed to the narrowest part of the chamber, where the air passes the germicidal bulbs, and the entrained germs are killed and the air is purified to the extent as previously reviewed. The UVB bulbs emit a minimum of cold ozone into the airstream, but sufficient enough to sterilize the bacteria and to achieve some odor control, without emitting much in the way of cold ozone into the ambient atmosphere, after the air is treated and forced back into the proximate room.

All of the operative components necessary to achieve the operations of this apparatus are self-contained in the housing, such as blowers, transformers, switching, electrical lines, and whatever other circuitry that is required to achieve the effective operations of this air purifier.

The apparatus can be formed of varying sizes, depending upon the capacities of air treatment desired and required, and it may include a single, or double blower, or even more, depending upon the circumstances of its usage.

By way of background, it is known that air contamination and pollution is a long standing problem which affects everyone to varying degrees. In urban areas, pollution levels continue to rise. Even in rural areas, contaminants, such as mold, pollen, and bacteria exist in the air. Air contamination presents a serious long term health risk to many. Contaminants also afflict persons suffering from allergies, asthma, emphysema, and many more respiratory related illnesses.

Frequently, filters have been used to remove particular contaminants, such as smoke, dust, pollen, and lint. Filters are designed to physically filter out such contaminants, usually of a specified size, or larger.

It is also known that ozone can break down other contaminants, such as the viral type, bacteria, and mold spores. Ozone having a high energy level, such as photozone, breaks up gasses and odors, and destroys pathogens.

Others have attempted with varying degrees of success to devise methods and devices for improving air quality by creating ozone. Such prior art methods and devices suffer significant disadvantages.

It is known that applying a substantial voltage between two points creates sparking, similar to lightning. The sparking reacts with ambient air to create ozone. However, this method creates a serious health risk. The heated sparks cause heated nitrogen gas to form. The heated nitrogen gas reacts with the ambient air to form nitric oxide. When nitric oxide is ingested by humans, or animals, for that matter, moisture from the lung causes the nitric oxide to turn into nitric acid. Nitric acid can cause many adverse reactions.

Another known method for creating ozone employs polarized plates, one positively charged and the other negatively charged. The electric field between the polarized plates creates ozone. However, contaminants deposit on the plates over time and cause sparks which cause nitrous gases to form with the same adverse effects as described.

It is also known that ozone can be created without forming nitrous gases by eliminating the use of heat, known as the cold process. By this method, ozone can be created by subjecting air or water to high frequency electromagnetic energy, or ultraviolet (UV) light. The amount and type of ozone created depends on the wavelength and intensity of the energy source. Such a light source, such as a UV lamp, emits a stream of invisible packets or compressions of energy, referred to as photons or waves. The energy carried by photons increases as the wave length shortens.

Systems have been employed to create ozone by the cold process, thereby avoiding nitrous gas emissions. Such systems are intended for use in homes, hospitals, and other areas where it is desirable to remove contaminants. To create ozone, or photozone, by the cold process, blowers must be employed to draw air into the system and direct the air past a light source, for example, a UV lamp.

It is, therefore, the principal object of this invention to provide an air purification system that effectively utilizes ultraviolet light for treatment of air passing through a confined area to achieve its decontamination, before the treated air is passed back into a room or other facility.

Another object of this invention is to provide an air purification system utilizing ultraviolet light.

Another object of this invention is to provide an air purification system that is of compact size, and through the use of prearranged deflectors, can immediately absorb untreated air into its housing, pass it through a confined ozone segment, and direct the treated and purified air back into the room.

Still another object of this invention is to provide an air purification housing that may be used in laboratory or hospital settings to reduce and eliminate the contaminants viral matter and bacteria from such locations.

Yet another object of this invention is to provide an air purification system that effectively processes air in a manner that provides for its full exposure to ultraviolet light, to decrease bacteria and infectious contaminants, before returning the circulating air back into the confined space.

Still another object of this invention is to provide an air purification system that can work just as effectively in a residence, as it will in a hospital room setting, or even in an airplane.

Still another object of this invention is to provide an air purification system that generates no harmful gases.

Yet another object is to provide an air purification apparatus that is of very low maintenance.

Another object of this invention is to provide an air purification system that is inexpensive and efficient of operation.

These and other objects may become more apparent to those skilled in the art upon review of the summary of the invention as provided herein, and upon undertaking a study of the description of its preferred embodiment, in view of the drawings.

### BRIEF DESCRIPTION OF THE DRAWINGS

In referring to the drawings,

FIG. 1 is an isometric view of the model of the air purification system and apparatus of this invention;

FIG. 2 is a view internally of the apparatus with its front cover removed;

FIG. 3 is a side view of the internal aspects of the apparatus;

FIG. 3a is a front view of the deflector;

FIG. 3b is a side view of the deflector;

FIG. 4 is a front view of the cover for the apparatus;

FIG. 5 is a side view of the cover shown the arranged filter at its lower segment;

FIG. 6 is an isometric view of a smaller model of the air purification system and apparatus of this invention;

FIG. 7 is a back view of the apparatus;

FIG. 8 is an internal side view showing the location of the blower and the upper air passage; and

FIG. 9 is an internal view of the housing showing the location of its intake, and exhaust.

### DESCRIPTION OF THE PREFERRED EMBODIMENTS

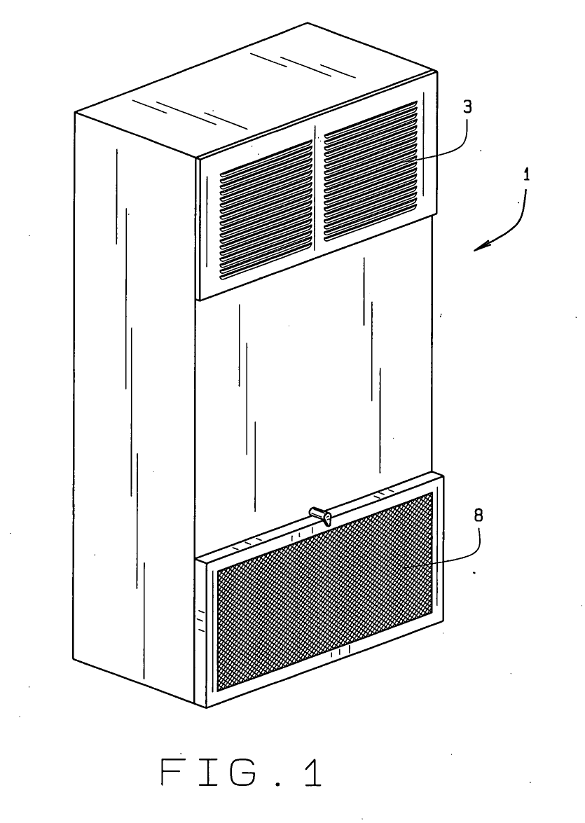

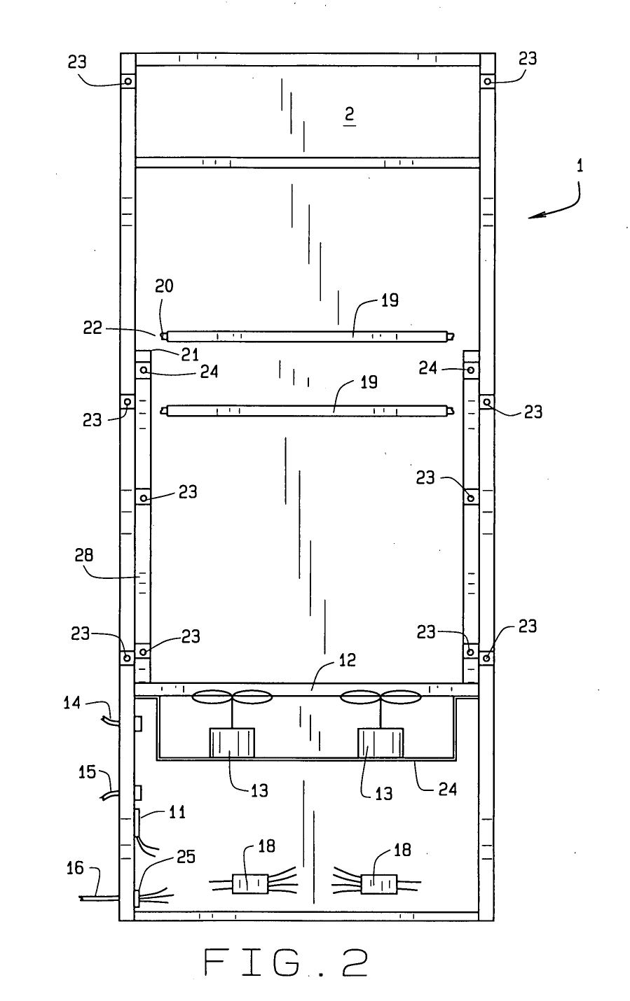

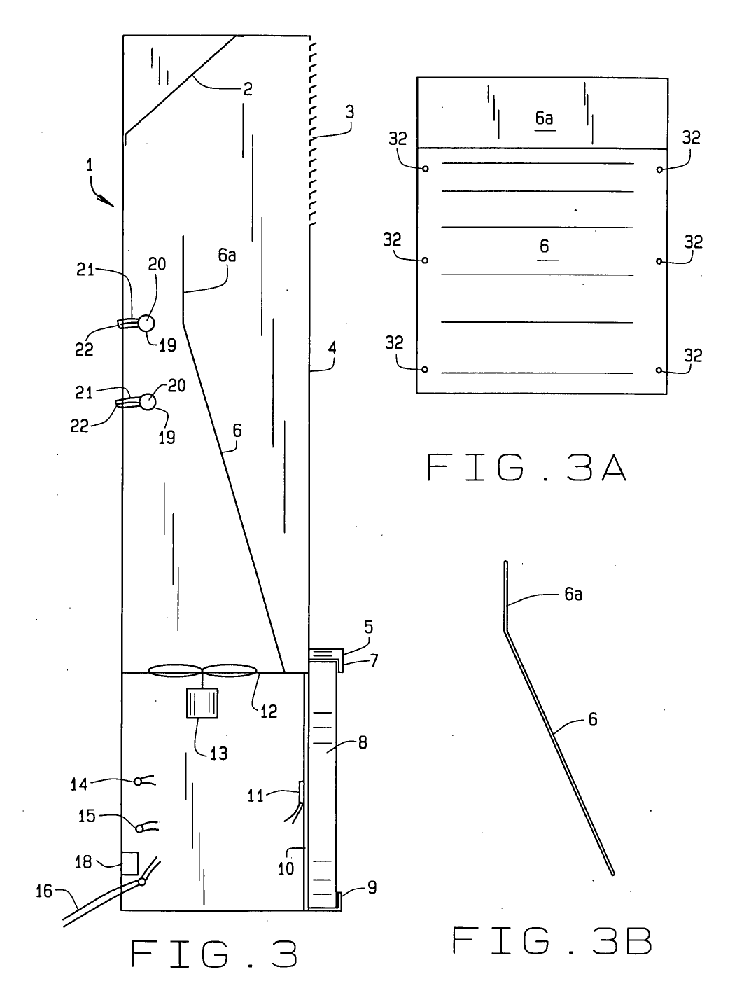

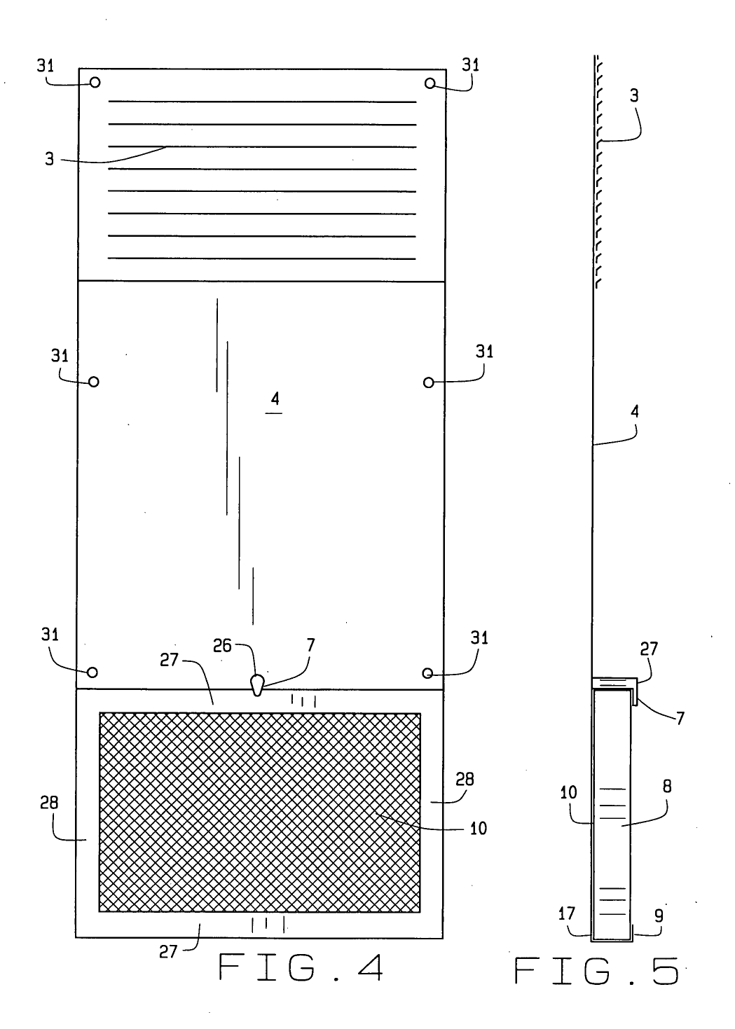

In referring to the drawings, and in particular FIG. 1, therein is shown the air purification system and apparatus 1 of this invention. It includes a housing, as can be noted, with a filtered intake, as at 8, provided at its bottom, and with the exhaust or outlet 3 provided in its upper region. As can be noted in FIGS. 2 and 3, there is a baffle 2 which is arranged at a forty-five degree angle, in the upper region internally of the housing, and at that location the baffle provides for direction of the treated air towards the front of the cabinet, for discharge out of its exhaust grill 3, as can be noted. There is also a front cover provided upon the housing 1, and the cover 4 includes an upper segment, with the exhaust grill 3 provided at its uppermost region, and the filter 8 being provided at its lower segment, and which fits within the housing, when the cover is applied. This can be seen also in FIGS. 4 and 5. A lock and screw 5 is provided for holding the filter in position, and the filter and the front cover fit upon a front cover lip 9, as can be noted. The lock screw 5 cooperates with the filter latch 7, for holding these components in place. The front cover 4 is actually fastened by screws to the housing, during its installation. A protective grill 10 is furnished for overlying the filter element, when installed.

Provided internally of the housing is a deflector 6, which is widened at its lower segment, for cooperating with the blower 13, to allow for the air to pass into the region between the deflector 6, and the back wall of the housing, up to its narrowest point, as can be noted at 6a. It is at this location where there is a confined and constricted space between the deflector 6, and its narrowest point at 6a, and the back wall of the apparatus, so that the air passing therethrough is at it narrowest location and having direct exposure to the ultraviolet light, as at bulbs 19, to attain its maximum effect. At this region, all of the air will be somewhat condensed when it passes by the region of the UV bulbs 19, which are supported by their sockets 20, and the spacers 21 fastened by screws 22 to the back wall of the housing 1. Thus, as the air passes up through the housing, it is condensed by the deflector 6, into the region of the UV bulbs, where the ultraviolet emitted effectively reduces any pathogens or any other bacteria that may be in the air, to eliminate it, for sending purified air against the deflector 2 for advancement out of the front exhaust grill 3 of the housing, during application.

FIGS. 3a and 3b show the configuration of the deflector 6, and its shape in configuration for locating within the apparatus 1.

In the lower segment of the housing, there is a cabinet divider 12, that supports the blower (s) 13, that attracts the air in from the room, through the filter 8, and directs it upwardly into the confinement space formed by the deflector 6, as can be seen.

Other elements provided within the lower segment of the housing include a safety switch 11, various high and low switches 14, for turning on the second UV light, if necessary, and a power on and off switch 15, as can be seen. An electric cord 16 supplies power to the electrically operative components, such as the blower 13, through its switching mechanism, and also to the ballast 18, for providing reduced voltage to the lights to attain their proper operations.

Each of the lamps provided within the structure of this housing, comprising the ultraviolet lamps as defined, may be the type of lamps that are in the region of approximately twelve (12) inches in length, and which operate off of approximately fourteen watts. Their approximate current and milliamps is 425. Total watts consumed are approximate 3.7. The ozone output is approximately 1. The effective hours of lamp life is 10,000. These types of lamps are available from a Company named Ster-L-Ray, of Hauppauge, N.Y., and are sold under catalog number GPH287T5L-GPH287T5VH.

As can also be seen in the structure for the housing of this invention, in FIG. 4, there is a fastening latch, as at 26, that holds the filter 8 in place. The cabinet front cover 4 is held into position on the housing by means of a series of fasteners, such as screws, provided through the apertures 31.

It may also be commented that a carbon filter may be utilized in combination with the filter element 8, in order to filter out any smoke that is drawn into the housing, during operations of the air purification system of this invention.

The various figures show the location of filters, such as the filter element 8, the filter 6 of FIG. 7, and related filters. The various filters shown preferably comprise a combination of a standard filter, in addition to a carbon filter, to add any additional advantage in the manner of furnishing smoke removal, from the apparatus, during its operations.

During usage, the ambient air is drawn in through the static charge filter 8, the filter incorporates a germicidal treated media, that will attract and collect seventy-five to eighty percent of the airborne pollutants. The filter is secured to the front panel by its latching mechanism, and behind the filter is the protective grill to stop any debris from entering into the housing. The filtered air is moved through the housing at approximately 150 CFM's, by each of the twin blowers 13, as can be seen in FIG. 2. The air is then forced upwardly and compacted into the UV chamber, in the proximity of the lamps 19, as can be noted. As stated, the UV chamber, due to the upper segment 6a of the deflector forms a chamber of approximately fourteen inches by two to three inches in cross-section. The UV germicidal bulbs 19 are spaced approximately three inches apart, one above the other, and held in place by means of the mechanical means and sockets, as described. It is to be noted that there is direct exposure to the UV bulbs, or their ultraviolet light, as they operate within a housing, since they are located behind the deflector 6, and its upper segment 6a, so any occupant of the room or other facility is just not exposed to the ultraviolet light. As the air passes through this condensed area, this is where the germs are killed and the air is purified. Also, the UVB bulb 19 emits a minimum cold zone ozone into the airstream. Because of the closeness the UVB bulb, as at 19, the ultraviolet light can purify the air at a maximum. The reason for using the UVB bulb, is that it emits some amount of cold ozone, because ozone is an excellent sterilizer for bacteria and odor control. The air leaves the UV chamber and impinges upon the forty-five degree baffle 2, directing the air forwardly and out the front exhaust grill 3 of the housing.

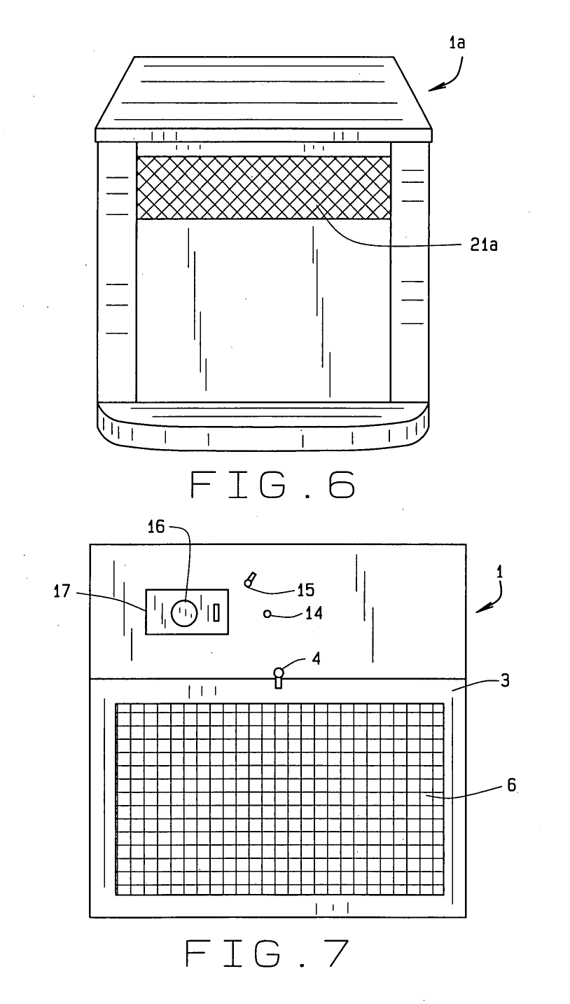

A smaller model and modified air purification system and apparatus is shown in FIG. 6. The housing 1a is formed of a rectangular design, as can be seen in FIG. 9, and internally thereof has a series of rails 2a secured to the internal side walls of the formed housing.

The housing includes a filter element 3a, which may be approximately nine to ten inches in height, and fourteen inches in width. Various latches 4a and 5a are provided for holding the filter in place. A grill 6a is provided internally of the filter, to help hold it in position, and to prevent any enlarged debris from entering into the housing.

As noted in FIG. 7, this provides a back view of the apparatus, and as shown are a series of control mechanisms, such as the power on and off switch 15, align cord 14 extends from the interior of the unit, and which conducts electricity to the operative controls. A twenty-four (24) hour timer with override switch and multiple on and off setting, of twenty (20) minutes to several hours, is shown at 16. Finally, FIG. 17 shows the timer mounting screw holes. More specifically, the power controls are arranged at the upper segment of the model, on the back side, but readily available for convenient usage and operation.

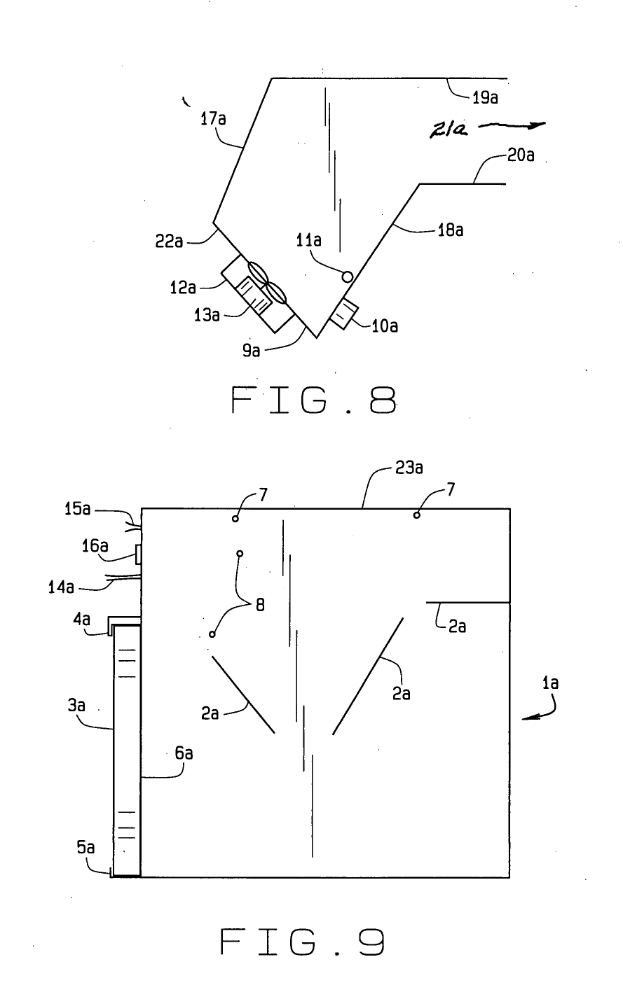

As can be seen in FIG. 8, a chamber for flow of air through the housing is provided, as at 9a, it may be made of stainless steel, and is shaped to the configuration shown having a series of plenum walls 17a and 18a, which mount onto the rails 2a, as previously defined. These plenum walls 17a and 18a act as deflectors for guidance of the treated air upwardly, and through the integral passage formed by the upper and lower walls 19a and 20a, as can be seen. Thus, by directing the treated air in these directions, it then passes through the exhaust outlet 21a provided at the upper front of the housing, as can be noted.

The lower end of the air plenum, within the chamber 9a, is formed by a structured wall 22a, and it mounts the fan bracket 12a and the fan and motor 13a, as can be seen. This particular housing, through its operations of the fan and motor, is capable of passing 150 CFM's of air through the housing, and its air plenum 9a, during usage.

As can be noted, a ballast 10a secures to the wall 18a, and it is connected with the UVB bulb 11a, which treats and purifies the air directed into the plenum, by means of the operations of the fan 13a, during usage. In addition, a safety switch is located close to these power operative components, which provides a high/low switch at this vicinity, also, and which consolidates switches saving in the hook-up wiring and connectors used in the installation of these electrical components,

Thus, air is drawn directly into the housing by means of the fan 13a, through the filter 3a, it passes the air directly over the UVB bulb 11a during its illumination, and then passes the air upwardly and deflects it forwardly through the plenum 9a, and its upper and lower walls 19a and 20a, for exhaust out of the front grill 21a of the housing.

Other instrumentalities utilized to render this air purification system operative include the application of an electrical line cord 14a to the housing, an off and on switch 15a to render the system operative, and a timer 16a is coupled into the electrical circuitry to provide a twenty-four hour timer with an override switch multiple off and on setting at twenty minute intervals, up to several hours of operation.

The housing 1a may be formed with one of its walls, or an upper lid, such as at 23a, which may be removed for attaining access into the housing to provide for its servicing, and cleaning, if necessary.

This describes the structure and operations of the various models for the air purification system and apparatus of this invention. As explained, the smaller embodiment, as just described, may provide an approximate 150 cubic feet per minute flow of air through the housing, during its operations, while the larger two blower models may pass approximately up to 300 CFM's of air through its housing during its functioning. This is just a matter of size, as to what capacity is desired for the facility, to attain the most effective air purification through the use of this system.

Variations or modifications to the subject matter of this invention may occur to those skilled in the art upon review of the development as described herein. Such variations, within the spirit of this development, are intended to be encompassed within the scope of the invention as described. The depiction of the invention in the preferred embodiment, and its disclosure in the drawings, are set forth for illustrative purposes only.

1. An air purification system including a housing, said housing being fabricated from various side, front, back, top and bottom walls, one of the back and front walls at their lower segments having an air intake, a filter operatively associated with the air intake, a series of deflectors provided within the housing, at least one blower mounted upon the deflectors, the blower drawing air into the housing through the air intake, and passing it into the air plenum formed by the deflectors, the deflectors confining and constricting the passing air into a condensed space, at least one ultraviolet lamp provided within the constricted space of the air plenum, and which when illuminated, providing for elimination of any bacteria and viral matter entrained within the condensing air, to provide for its purification, said deflectors aligned with an exhaust outlet provided in one of the walls of the housing, for transferring and discharging more purified air back into any room in which the air purification system locates.

2. The air purification system of claim 1 and wherein the constricted area of the formed air plenum having dimensions of approximately 2″×14″ in cross section, and the at least one ultraviolet lamp being provided within that constricted space of the formed air plenum.

3. The air purification system of claim 2 and including said filter being located within the front wall of the housing.

4. The air purification system of claim 2 wherein said filter being located within the said back wall of the housing.

5. The air purification system of claim 1 and wherein said side walls include a series of rails connecting thereto, said air plenum incorporating a blower installed within the housing by mounting onto the located rails and the at least one ultraviolet lamp being located in proximity with the air blower within the formed air plenum to provide for purification of the air passing through the system.

6. The air purification system of claim 5 and wherein said air blower locates proximate the intake of the housing.

7. The air purification system of claim 5 wherein said air blower locates proximate the exhaust outlet of the housing.

8. The air purification system of claim 2 wherein a deflector locates within the housing, said deflector having a bend at its upper end, the bend of the air deflector locating in proximity with the back wall of the housing, to thereby form a confining and constricting location for passage of the air through the formed constricted space.

9. The air purification system of claim 5 and including a timer operatively associated with the housing and providing for the timed operations of the system during usage.

10. The air purification system of claim 1 and including a pair of blowers mounted upon the deflectors.

11. The air purification system of claim 1 and including two ultraviolet lamps provided within the constricted space of the air plenum.

12. A compact air purification system including a housing, said housing being fabricated from various side, front, back, top and bottom walls, one of the back and front walls at their lower segments having an air intake, a filter operatively associated with the air intake, said side walls including a series of rails connecting thereto, an air plenum incorporating a blower installed at its intake for the air plenum within the housing by mounting onto the located rails, and at least one ultraviolet lamp being located in proximity with the air blower within the formed air plenum, to provide for full exposure and purification of the passing air through the air plenum to provide for the elimination of any bacteria and viral matter entrained within the passing air during system operation, said air plenum forming an intake at the location of the incorporated blower, and said air plenum forming an exhaust outlet providing for the discharge of the purified air through the front wall of the housing.
